(19) United States
(12) Patent Application Publication
(10) Pub. No.: US 2022/0002360 A1
(43) Pub. Date: Jan. 6, 2022

(54) EXTRACELLULAR MATRIX PROTEIN COMPOSITIONS AND METHODS FOR TREATING WOUNDS

(71) Applicant: SMSBIOTECH, INC., San Marcos, CA (US)

(72) Inventor: Abdulkader Rahmo, San Marcos, CA (US)

(21) Appl. No.: 17/279,408

(22) PCT Filed: Sep. 11, 2019
(86) PCT No.: PCT/US2019/050655

§ 371 Date: Mar. 24, 2021

Related U.S. Application Data
(60) Provisional application No. 62/738,253, filed on Sep. 28, 2018.

(51) Int. Cl. C07K 14/47; C12N 5/0789

(52) U.S. Cl. CPC C07K 14/47; C12N 2537/10; C12N 2533/90; C12N 5/0647

(57) ABSTRACT

Disclosed herein are compositions comprising extracellular matrix proteins produced by small mobile stem cells, and systems comprising the composition. Also disclosed are methods of making the compositions and systems, and methods of using the compositions for treating or ameliorating a wound, such as a diabetic ulcer, a venous ulcer, a chronic ulcer, or a pressure ulcer, in a subject.

### CROSS-REFERENCE TO RELATED APPLICATIONS

This application claims the benefit of priority to U.S. Provisional Application No. 62/738,253, filed Sep. 28, 2018, the disclosure of which is hereby expressly incorporated by reference in its entirety.

### FIELD

Aspects of the present disclosure relate generally to compositions and systems comprising extracellular matrix proteins produced from small mobile stem cells. The disclosure also relates to methods of making said compositions and systems, and methods of using the compositions for treating or ameliorating a wound.

### BACKGROUND

Stem cells are immature, unspecialized cells capable of renewing themselves for extended periods through cell division. Under certain conditions, they differentiate into mature, functional cells.

The use of stem cells and stem cell derivatives is currently of great interest to medical research, particularly for the prospects of providing reagents for treating tissue which has been damaged by various causes such as genetic disorders, injuries, and/or disease processes.

Small mobile stem (SMS) cells have recently been isolated and characterized, for example in WO 2014/200940, hereby expressly incorporated by reference in its entirety. SMS cells are adherent cells that are from 4.5 to 5.5 μm in diameter, and are obtained from sources such as umbilical cord, peripheral blood, bone marrow, or solid tissue. SMS cells may be used to produce extracellular matrix (ECM) and/or ECM proteins in tissue culture, as described in WO 2017/172638, hereby expressly incorporated by reference in its entirety.

ECM is a structure, scaffold, or platform made up of a chemically or biochemically defined material to which various cells (e.g, dermal, muscle, nerve, connective tissue, fascia, dura or peritoneum) of higher vertebrates can adhere to and multiply without causing toxicity or inhibition of cell replication. Components of ECM can include proteoglycans, glycoproteins, non-proteoglycan saccharides, and fibers.

ECM is essential for regrowth and healing of tissue. In human fetuses, for example, the ECM works with stem cells to grow and regrow all parts of the human body, and fetuses can regrow tissue damaged in the womb. However, after reaching full development, the ECM stops functioning.

ECM is also useful in medical applications, where it is used for injury repair and tissue engineering by preventing or inhibiting the immune system from responding to the injury with inflammation. In addition, the ECM facilitates surrounding cells to repair the tissue instead of forming scar tissue. In these cases, the ECM is often extracted from a source, such as from pig bladders, decellularized, and used in the given application. ECM powders can be used for tissue engineering, for example.

Wound healing is a complex cascade of events that attempts to maintain homeostasis in the wounded tissue. Closing of the wound is essential to the healing process as it protects the tissue itself from infection with foreign agents, such as opportunistic bacterial pathogens. Biofilms established by these pathogens are a common cause of chronic infections that slow or prevent wound healing. The bacterial biofilms themselves are challenging to eliminate with conventional antibiotics due to an extensive exopolymeric layer covering the pathogen that limits diffusion of these drugs into the biofilm.

Currently, the clinical treatment of wounds presents a significant challenge. Therapies for the treatment of wounds include autologous tissue grafts and fat transplantation (replacing traumatized tissue with a patient's own skin and fat tissue, taken from a distant site), and alloplastic (synthetic) implants. However, these methods present significant problems for the patient including donor site morbidity, implant migration, rupture, volume reduction, and foreign body reaction. The need for additional medicaments to facilitate wound healing is manifest.

### SUMMARY

The present disclosure relates generally to compositions and systems comprising extracellular matrix or extracellular matrix proteins isolated from small mobile stem (SMS) cells and methods of making and using the same. Accordingly, aspects of the invention include the use of extracellular matrix or extracellular matrix proteins isolated from a culture of SMS cells as a medicament or use of extracellular matrix or extracellular matrix proteins isolated from a culture of SMS cells to treat or ameliorate a wound, such as a diabetic ulcer, a venous ulcer, a chronic ulcer, or a pressure ulcer in a subject e.g., a human.

Some embodiments provided herein relate to compositions comprising an extracellular matrix (ECM) protein or ECM derived from SMS cells in culture. In some embodiments, the SMS derived ECM or ECM protein comprises at least one cross-link, which has been induced or formed by a cross-linking agent or technique e.g., radiation, chemical, mechanical, or temperature. In some embodiments, the cross-linked ECM or ECM protein is denatured. In some embodiments, the at least one cross-link is induced by radiation, such as gamma radiation, ultra violet light, or electron beam radiation (e-beam radiation). In some embodiments, the cross-link is induced by exposure to a chemical, or a physical cross-linking environment, such as an acid, base, or a shearing force. In some embodiments, the cross-linked ECM or ECM protein comprises a modification, such as, for example, an acetylation, acylation, carboxylation, glycosylation, hydroxylation, lipidation, methylation, pegylation, phosphorylation, prenylation, sulfation, or ubiquitination. In some embodiments, the cross-linked ECM or ECM protein comprises agrin, filaggrin, mucin, secreted phosphoprotein 24 (bone matrix), nidogen, cadherins, clathrin, collagen, defensin, elastin, entactin, fibrillin, fibronectin, vitronectin, keratin, laminin, microtubule-actin cross-linking factor 1, SPARC-like protein, nesprin (nesprin-1, nesprin-2, nesprin-3), fibrous sheath-interacting protein, myomesin, nebulin, keratinocyte proline rich protein, plakophilin, integrin, exportins, transportin, tenascin, perlecan, sortilin-related receptor, tensin, or titin, or total protein, or a fragment of any one or more of the aforementioned proteins. In some embodiments, the cross-linked ECM or ECM protein comprises an anti-microbial compound, such as a bacterial, yeast, or fungi inhibiting compound. In some embodiments, the anti-microbial compound is selected from the group consisting of dermicidin, collectin, C-type lectin family 4, septin 12, defensin, and pancreatic ribonuclease. In some embodiments, the composition is formulated as an implant, a powder, an aerosol, a cream, an emulsion, a foam, a foamable liquid, a gel, a lotion, an ointment, a paste, a salve, a serum, a solution, or a spray. In some embodiments, the composition further comprises one or more antimicrobial agents, antibiotics, anti-inflammatory compounds, or an analgesic not natively present in said cross-linked SMS-derived ECM or ECM protein. In some embodiments, the composition further comprises cells such as fibroblasts, endothelial cells, keratinocytes, melanocytes, or stem cells. In some embodiments, the composition further comprises collagen, polygalactin mesh, polylactic-glycolic acid, polycaprolactone, polypyrrole, hyaluronan, chitosan, or xenogeneic tissue. In some embodiments, the composition further comprises a growth factor, such as bone morphogenetic protein, insulin like growth factor, osteoclast stimulating factor, insulin like growth factor binding protein, calmodulin like protein, or thymosin.

Some embodiments provided herein relate to cosmetic compositions. In some embodiments, the cosmetic comprises an ECM or ECM protein derived from a culture of SMS cells. In some embodiments, the SMS-derived ECM or ECM protein in the cosmetic comprises at least one cross-link, which has been induced by exposure to a cross-linking agent or a cross-linking technique e.g., radiation, chemical, mechanical, or temperature. In some embodiments, the cross-linked ECM or ECM protein in the cosmetic is denatured. In some embodiments, the at least one cross-link is induced by radiation, such as gamma radiation, ultra violet light, or electron beam radiation (e-beam radiation). In some embodiments, the cross-link is induced by exposure to a chemical, or a physical cross-linking environment, such as an acid, base, or a shearing force. In some embodiments, the cross-linked ECM or ECM protein in the cosmetic comprises a modification, such as, for example, an acetylation, acylation, carboxylation, glycosylation, hydroxylation, lipidation, methylation, pegylation, phosphorylation, prenylation, sulfation, or ubiquitination. In some embodiments, the cross-linked ECM or ECM protein comprises agrin, filaggrin, mucin, secreted phosphoprotein 24 (bone matrix), nidogen, cadherins, clathrin, collagen, defensin, elastin, entactin, fibrillin, fibronectin, vitronectin, keratin, laminin, microtubule-actin cross-linking factor 1, SPARC-like protein, nesprin (nesprin-1, nesprin-2, nesprin-3), fibrous sheath-interacting protein, myomesin, nebulin, keratinocyte proline rich protein, plakophilin, integrin, exportins, transportin, tenascin, perlecan, sortilin-related receptor, tensin, or titin, or total protein, or a fragment of any one or more of the aforementioned proteins. In some embodiments, the cross-linked ECM or ECM protein in the cosmetic comprises an anti-microbial compound, such as a bacterial, yeast, or fungi inhibiting compound. In some embodiments, the anti-microbial compound is selected from the group consisting of dermicidin, collectin, C-type lectin family 4, septin 12, defensin, and pancreatic ribonuclease. In some embodiments, the composition is formulated as an implant, a powder, an aerosol, a cream, an emulsion, a foam, a foamable liquid, a gel, a lotion, an ointment, a paste, a salve, a serum, a solution, or a spray. In some embodiments, the cosmetic further comprises one or more antimicrobial agents, antibiotics, anti-inflammatory compounds, or an analgesic not natively present in said SMS-derived cross-linked ECM or ECM protein. In some embodiments, the cosmetic further comprises cells such as fibroblasts, endothelial cells, keratinocytes, melanocytes, or stem cells. In some embodiments, the cosmetic further comprises collagen, polygalactin mesh, polylactic-glycolic acid, polycaprolactone, polypyrrole, hyaluronan, chitosan, or xenogeneic tissue. In some embodiments, the cosmetic further comprises a growth factor, such as bone morphogenetic protein, insulin like growth factor, osteoclast stimulating factor, insulin like growth factor binding protein, calmodulin like protein, or thymosin. In some embodiments, the cosmetic further comprises a fragrance, emollient, or fatty acid, such as oleic acid or palmitoleic acid.

Some embodiments provided herein relate to methods of making the compositions described herein. In some embodiments, the composition comprises an extracellular matrix (ECM) or ECM protein isolated from a culture of SMS cells. In some embodiments, the ECM or ECM protein comprises at least one cross-link, which has been induced by exposure to a cross-linking agent or a cross-linking technique e.g., radiation, chemical, mechanical, or temperature. In some embodiments, the cross-linked ECM or ECM protein is denatured. In some embodiments, the at least one cross-link is induced by radiation, such as gamma radiation, ultra violet light, or electron beam radiation (e-beam radiation). In some embodiments, the cross-link is induced by exposure to a chemical, or a physical cross-linking environment, such as an acid, base, or a shearing force. In some embodiments, the cross-linked ECM or ECM protein comprises a modification, such as, for example, an acetylation, acylation, carboxylation, glycosylation, hydroxylation, lipidation, methylation, pegylation, phosphorylation, prenylation, sulfation, or ubiquitination. In some embodiments, the cross-linked ECM or ECM protein comprises agrin, filaggrin, mucin, secreted phosphoprotein 24 (bone matrix), nidogen, cadherins, clathrin, collagen, defensin, elastin, entactin, fibrillin, fibronectin, vitronectin, keratin, laminin, microtubule-actin cross-linking factor 1, SPARC-like protein, nesprin (nesprin-1, nesprin-2, nesprin-3), fibrous sheath-interacting protein, myomesin, nebulin, keratinocyte proline rich protein, plakophilin, integrin, talins, exportins, transportin, tenascin, perlecan, sortilin-related receptor, tensin, titin, total protein, or a fragment of any one or more of the aforementioned proteins. In some embodiments, the cross-linked ECM or ECM protein comprises an anti-microbial compound, such as bacterial, yeast, or fungi inhibiting compound. In some embodiments, the anti-microbial compound is selected from the group consisting of dermicidin, collectin, C-type lectin family 4, septin 12, defensin, and pancreatic ribonuclease. In some embodiments, the composition is formulated as an implant, a powder, an aerosol, a cream, an emulsion, a foam, a foamable liquid, a gel, a lotion, an ointment, a paste, a salve, a serum, a solution, or a spray. In some embodiments, the composition further comprises one or more antimicrobial agents, antibiotics, anti-inflammatory compounds, or analgesics not natively present in said cross-linked ECM or ECM protein. In some embodiments, the composition further comprises cells such as fibroblasts, endothelial cells, keratinocytes, melanocytes, or stem cells. In some embodiments, the composition further comprises collagen, polygalactin mesh, polylactic-glycolic acid, polycaprolactone, polypyrrole, hyaluronan, chitosan, or xenogeneic tissue. In some embodiments, the composition further comprises a growth factor, such as bone morphogenetic protein, insulin like growth factor, osteoclast stimulating factor, insulin like growth factor binding protein, calmodulin like protein, or thymosin. In some embodiments, the composition is a cosmetic. In some embodiments, the cosmetic further comprises a fragrance, emollient, or fatty acid, such as oleic acid or palmitoleic acid. In some embodiments, the methods of making comprise culturing a population of small mobile stem (SMS) cells for a time sufficient to form an ECM or ECM protein. In some embodiments, the ECM or ECM protein adheres to a culture vessel. In some embodiments, the ECM or ECM protein is present in the culture media e.g., liberated from adherence to the culture vessel or floating. In some embodiments, the method of making comprises removing the ECM protein from the culture vessel or media to obtain an isolated ECM or ECM protein. In some embodiments, the method comprises washing the isolated ECM or ECM protein. In some embodiments, the method comprises drying the ECM or ECM protein. In some embodiments, the ECM or ECM protein is dried into a powder. In some embodiments, the method or making further comprises introducing at least one cross-link into said ECM or ECM protein such as by exposure to a radiation, preferably gamma radiation, ultra violet light radiation, x-ray radiation, or electron beam radiation (c-beam radiation). In some embodiments, the method further comprises introducing at least one cross-link into said ECM or ECM protein by exposure to a chemical, or a physical cross-linking environment, such as an acid, base, or a shearing force. In some embodiments, the method further comprises freezing the ECM or ECM protein. In some embodiments, the method further comprises joining the ECM or ECM protein to a support or surface e.g., surface of a wound dressing or culture vessel. In some embodiments, the support comprises a cellulose or one or more sugars or sugar alcohols.

Some embodiments provided herein relate to systems for facilitating wound healing. In some embodiments, the systems comprise a wound dressing material comprising a composition as described herein. In some embodiments, the wound dressing material comprises a bandage, a wipe, a gauze, a sponge, a mesh, a pad, an adhesive bandage, a nylon, or an absorbent wound dressing material. In some embodiments, the composition comprises an extracellular matrix (ECM) or ECM protein isolated from a culture of SMS cells. In some embodiments, the isolated ECM or ECM protein comprises at least one cross-link, which has been induced by exposure to a cross-linking agent or a cross-linking technique e.g., radiation, chemical, mechanical, or temperature. In some embodiments, the cross-linked ECM or ECM protein is denatured. In some embodiments, the at least one cross-link is induced by radiation, such as gamma radiation, ultra violet light, or electron beam radiation (e-beam radiation). in some embodiments, the cross-link is induced by exposure to a chemical, or a physical cross-linking environment, such as an acid, base, or a shearing force. In some embodiments, the cross-linked ECM or ECM protein comprises a modification, such as, for example, an acetylation, acylation, carboxylation, glycosylation, hydroxylation, lipidation, methylation, pegylation, phosphorylation, prenylation, sulfation, or ubiquitination. In some embodiments, the cross-linked ECM or ECM protein comprises agrin, filaggrin mucin, secreted phosphoprotein 24 (bone matrix), nidogen, cadherins, clathrin, collagen, defensin, elastin, entactin, fibrillin, fibronectin, vitronectin, keratin, laminin, microtubule-actin cross-linking factor 1, SPARC-like protein, nesprin (nesprin-1, nesprin-2, nesprin-3), fibrous sheath-interacting protein, myomesin, nebulin, keratinocyte proline rich protein, plakophilin, integrin, talins, exportins, transportin, tenascin, perlecan, sortilin-related receptor, tensin, or thin, or total protein, or a fragment of any one or more of the aforementioned proteins. In some embodiments, the cross-linked ECM or ECM protein comprises an anti-microbial compound, such as bacterial, yeast, or fungi inhibiting compound. In some embodiments, the anti-microbial compound is selected from the group consisting of dermicidin, collectin, C-type lectin family 4, septin 12, defensin, and pancreatic ribonuclease. In some embodiments, the composition is formulated as an implant, a powder, an aerosol, a cream, an emulsion, a foam, a foamable liquid, a gel, a lotion, an ointment, a paste, a salve, a serum, a solution, or a spray. In some embodiments, the composition further comprises one or more antimicrobial agents, antibiotics, anti-inflammatory compounds, or analgesics not natively present in said cross-linked ECM or ECM protein. In some embodiments, the composition further comprises cells such as fibroblasts, endothelial cells, keratinocytes, melanocytes, or stem cells. In some embodiments, the composition further comprises collagen, polygalactin mesh, polylactic-glycolic acid, polycaprolactone, polypyrrole, hyaluronan, chitosan, or xenogeneic tissue. In some embodiments, the composition further comprises a growth factor, such as bone morphogenetic protein, insulin like growth factor, osteoclast stimulating factor, insulin like growth factor binding protein, calmodulin like protein, or thymosin. In some embodiments, the composition is a cosmetic. In some embodiments, the cosmetic further comprises a fragrance, emollient, or fatty acid, such as oleic acid or palmitoleic acid. In some embodiments, the composition comprises a plurality of different ECM proteins and a plurality of polysaccharides. In some embodiments, the plurality of ECM proteins and the plurality of different polysaccharides comprise at least one of agrin, filaggrin, secreted phosphoprotein 24 (bone matrix), vitronectin, mucin, nidogen, cadherins, clathrin, collagen, defensin, elastin, entactin, fibrillin, fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, keratinocyte proline rich protein, tenascin, perlecan, sortilin-related receptor, tensin, titin, total protein, hyaluronic acid, cellulose, or a fragment, analogue, or derivative of any one or more of the aforementioned proteins and polysaccharides.

Some embodiments provided herein relate to methods of treating or ameliorating a wound, e.g., a chronic wound or ulcer, in a subject, such as a human, domestic animal, farm animal or companion animal. In some embodiments, the methods comprise contacting a wound of the subject with a composition as described herein or with a system as described herein. In some embodiments, the wound of the subject is contacted with a system that comprises a wound dressing material comprising a composition as described herein. In some embodiments, the wound dressing material comprises a bandage, a wipe, a gauze, a sponge, a mesh, a pad, an adhesive bandage, a nylon, or an absorbent wound dressing material. In some embodiments, the wound is contacted with a composition comprising an extracellular matrix (ECM) or ECM protein isolated from a culture of SMS cells. In some embodiments, the ECM or ECM protein isolated from a culture of SMS cells comprises at least one induced cross-link e.g., induced by radiation, chemical, mechanical, or temperature. In some embodiments, the cross-linked ECM or ECM protein is denatured. In some embodiments, the at least one cross-link is induced by radiation, such as gamma radiation, ultra violet light, or electron beam radiation (e-beam radiation). In some embodiments, the cross-link is induced by exposure to a chemical, or a physical cross-linking environment, such as an acid, base, or a shearing force. In some embodiments, the cross-linked ECM or ECM protein comprises a modification, such as, for example, an acetylation, acylation, carboxylation, glycosylation, hydroxylation, lipidation, methylation, pegylation, phosphorylation, prenylation, sulfation, or ubiquitination. In some embodiments, the cross-linked ECM or ECM protein comprises agrin, filaggrin, mucin, secreted phosphoprotein 24 (bone matrix), nidogen, cadherins, clathrin, collagen, defensin, elastin, entactin, fibrillin, fibronectin, vitronectin, keratin, laminin, microtubule-actin cross-linking factor 1, SPARC-like protein, nesprin (nesprin-1, nesprin-2, nesprin-3), fibrous sheath-interacting protein, myomesin, nebulin, keratinocyte proline rich protein, plakophilin, integrin, talins, exportins, transportin, tenascin, perlecan, sortilin-related receptor, tensin, titin, total protein, or a fragment of any one or more of the aforementioned proteins. In some embodiments, the cross-linked ECM or ECM protein comprises an anti-microbial compound, such as bacterial, yeast, or fungi inhibiting compound. In some embodiments, the anti-microbial compound is selected from the group consisting of dermicidin, collectins, C-type lectin family 4, septin 12, defensin, and pancreatic ribonuclease. In some embodiments, the composition is formulated as an implant, a powder, an aerosol, a cream, an emulsion, a foam, a foamable liquid, a gel, a lotion, an ointment, a paste, a salve, a serum, a solution, or a spray. In some embodiments, the composition further comprises one or more antimicrobial aunts, antibiotics, anti-inflammatory compounds, or an analgesics not natively present in said cross-linked ECM or ECM protein. In some embodiments, the composition further comprises cells such as fibroblasts, endothelial cells, keratinocytes, melanocytes, or stem cells. In some embodiments, the composition further comprises collagen, polygalactin mesh, polylactic-glycolic acid, polycaprolactone, polypyrrole, hyaluronan, chitosan, or xenogeneic tissue. In some embodiments, the composition further comprises a growth factor, such as bone morphogenetic protein, insulin like growth factor, osteoclast stimulating factor, insulin like growth factor binding protein, calmodulin like protein, or thymosin. In some embodiments, the composition is a cosmetic. In some embodiments, the cosmetic further comprises a fragrance, emollient, or fatty acid, such as oleic acid or palmitoleic acid. In some embodiments, the composition comprises a plurality of different ECM proteins and a plurality of polysaccharides. In some embodiments, the plurality of ECM proteins and the plurality of different polysaccharides comprise at least one of agrin, filaggrin, secreted phosphoprotein 24 (bone matrix), vitronectin, mucin, nidogen, cadherins, clathrin, collagen, defensin, elastin, entactin, fibrillin, fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, keratinocyte proline rich protein, tenascin, perlecan, sortilin-related receptor, tensin, titin, total protein, hyaluronic acid, cellulose, or a fragment, analogue, or derivative of any one or more of the aforementioned proteins and polysaccharides.

In some embodiments, the methods of treating or ameliorating a wound e.g., a chronic wound or ulcer, comprise topically applying one or the aforementioned compositions to the wound of a subject. In some embodiments, the wound is a partial thickness wound or a full thickness wound. In some embodiments, the wound is a surface wound or an internal wound. In some embodiments, the wound is skin damage, an abrasion, a contusion, a burn, an incision, a laceration, a penetration wound, a puncture wound, a sore, or an ulcer. In some embodiments, the ulcer is a diabetic ulcer, a venous ulcer, a chronic ulcer, or a pressure ulcer. In some embodiments, the treating or ameliorating the wound of the subject promotes fill thickness or partial thickness repair, accelerates wound healing, promotes wound closure, causes wound regression, increases epithelialization, increases epithelial layer thickness, increases granulation, increases granular layer thickness, increases collagen deposition, increases capillarization, enhances vascularization, enhances immune modulation, enhances cell migration, or enhances cell growth, or any combination thereof. In sonic embodiments, one or more of the aforementioned compositions is applied to a wound area of a subject in an amount of 0.01, 0.05, 0.1, 0.5, 1, 1.5, 2, 2.5, 5, 10, 25, 50, 100, 500 mg, or 1000 mg/cm² or within a range defined by any two of the aforementioned amounts. In some embodiments, one or more of the aforementioned compositions is applied to the wound of the subject at least once daily, once weekly, once monthly, or once yearly. In some embodiments, the composition is applied to the wound of the subject at least three times a day. In some embodiments, the composition is applied to the wound of the subject sequentially in 2, 3, 4, 5, 6, 7, 8, 9, 10, or more applications. In some embodiments, one or more of the compositions or systems described herein is applied to the wound for a period of 1, 2, 3, 4, 5, 6, 7, 8, 9, 10, 15, 20, 30 or more days, 1, 2, 3, 4, 5, 6, 7, 8, 9, 10, 11, 12 or more months, or 1, 2, 3, 4, 5, 10, or more years.

Accordingly, some aspects of the present invention relate to the following numbered alternatives:

1. A composition, such as a medicament, cosmetic, or wound dressing, comprising an extracellular matrix (ECM) or ECM protein isolated from a culture of small mobile stem (SMS) cells, wherein, preferably said ECM or ECM protein comprises at least one induced cross-link, which has been induced by exposure to a cross-linking agent or a cross-linking technique e.g., radiation, chemical, mechanical, or temperature.

2. The composition of alternative 1, wherein the ECM or ECM protein is denatured.

3. The composition of alternative 1 or 2, wherein said at least one cross-link is induced by radiation, such as gamma radiation, ultra violet light, or electron beam radiation (e-beam radiation).

4. The composition of alternative 1 or 2, wherein the cross-link is induced by exposure to a chemical, or a physical cross-linking environment, such as an acid, base, or a shearing force.

5. The composition of any one of alternatives 1-4, wherein said cross-linked ECM or ECM protein comprises an acetylation, acylation, carboxylation, glycosylation, hydroxylation, lipidation, methylation, pegylation, phosphorylation, prenylation, sulfation, or ubiquitination.

6. The composition of any one of alternatives 1-5, wherein the cross-linked ECM or ECM protein comprises agrin, filaggrin, mucin, secreted phosphoprotein 24 (bone matrix), nidogen, cadherins, clathrin, collagen, defensin, elastin, entactin, fibrillin fibronectin, vitronectin, keratin, laminin, microtubule-actin cross-linking factor 1, SPARC-like protein, nesprin (nesprin-1, nesprin-2, nesprin-3), fibrous sheath-interacting protein, myomesin, nebulin, keratinocyte proline rich protein, plakophilin, integrin, talins, exportins, transportin, tenascin, perlecan, sortilin-related receptor, tensin, titin, total protein, or a fragment of any one or more of the aforementioned proteins.

7. The composition of any one of alternatives 1-6, wherein the cross-linked ECM or ECM protein comprises an anti-microbial compound, such as bacterial, yeast, or fungi inhibiting compound.

8. The composition of alternative 7, wherein the anti-microbial compound is selected from the group consisting of dermicidin, collectin, C-type lectin family 4, septin 12, defensin, and pancreatic ribonuclease.

9. The composition of any one of alternatives 1-8, wherein the composition is formulated as an implant, a powder, an aerosol, a cream, an emulsion, a foam, a foamable liquid, a gel, a lotion, an ointment, a paste, a salve, a serum, a solution, or a spray.

10. The composition of any one of alternatives 1-9, further comprising one or more antimicrobial agents, antibiotics, anti-inflammatory compounds, or an analgesics not natively present in said cross-linked ECM or ECM protein.

11. The composition of any one of alternatives 1-10, further comprising cells such as fibroblasts, endothelial cells, keratinocytes, melanocytes, or stem cells.

12. The composition of any one of alternatives 1-11, further comprising collagen, polygalactin mesh, polylactic-glycolic acid, polycaprolactone, polypyrrole, hyaluronan, chitosan, or xenogeneic tissue.

13. The composition of any one of alternatives 1-12, further comprising a growth factor, such as bone morphogenetic protein, insulin like growth factor, osteoclast stimulating factor, insulin like growth factor binding protein, calmodulin like protein, or thymosin.

14. A cosmetic comprising an extracellular matrix (ECM) or ECM protein isolated from a culture of small mobile stem (SMS) cells, wherein, preferably said ECM or said ECM protein comprises at least one induced cross-link, which has been induced by exposure to a cross-linking agent or a cross-linking technique e.g., radiation, chemical, mechanical, or temperature, and, optionally, a fragrance, emollient, or fatty acid, such as oleic acid or palmitoleic acid.

15. A method of making a composition of any one of alternatives 1-14, comprising:
culturing a population of small mobile stem (SMS) cells for a time sufficient to form an extracellular matrix (ECM) or ECM protein, which adheres to a culture vessel or which is present in the culture media e.g., floating or liberated from adherence to the culture vessel;
isolating the ECM or ECM protein from the culture vessel to obtain an isolated ECM or ECM protein;
optionally, washing the isolated ECM or ECM protein; and
optionally, drying the ECM or ECM protein, e.g., into a powder.

16. The method of alternative 15, further comprising introducing at least one cross-link into said ECM or ECM protein such as by exposure to a radiation, preferably gamma radiation, ultra violet light radiation, x-ray radiation, or electron beam radiation (e-beam radiation).

17. The method of alternative 15, further comprising introducing at least one cross-link into said ECM or ECM protein by exposure to a chemical, or a physical cross-linking environment, such as an acid, base, or a shearing force.

18. The method of any one of alternatives 15-17, further comprising freezing the ECM or ECM protein.

19. The method of any one of alternatives 15-18, further comprising joining the ECM or ECM protein to a support.

20. The method of alternative 19, wherein the support cellulose, a sugar, or a sugar alcohol.

21. A system for wound healing comprising a wound dressing material comprising a composition of any one of alternatives 1-14.

22. The system of alternative 21, wherein the wound dressing material comprises a bandage, a wipe, a gauze, a sponge, a mesh, a pad, an adhesive bandage, a nylon, or an absorbent wound dressing material.

23. The system of any one of alternatives 21-22, wherein the composition comprises a plurality of different ECM proteins and a plurality of polysaccharides.

24. The system of alternative 23, wherein the plurality of ECM proteins and the plurality of different polysaccharides comprise at least one of agrin, filaggrin, secreted phosphoprotein 24 (bone matrix), vitronectin, mucin, nidogen, cadherins, clathrin, collagen, defensin, elastin, entactin, fibrillin, fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, keratinocyte proline rich protein, tenascin, perlecan, sortilin-related receptor, tensin, titin, total protein, hyaluronic acid, cellulose, or a fragment, analogue, or derivative of any one or more of the aforementioned proteins and polysaccharides.

25. A method of treating or ameliorating a wound comprising contacting a wound of a subject with the composition of any one of alternatives 1-14 or with the system of any one of alternatives 21-24.

26. The method of alternative 25, wherein the contacting comprises topically applying the composition to the wound of said subject.

27. The method of any one of alternatives 25-26, wherein the wound is a partial thickness wound or a full thickness wound.

28. The method of any one of alternatives 25-27, wherein the wound is a surface wound or an internal wound.

29. The method of any one of alternatives 25-27, wherein the wound is skin damage, an abrasion, a contusion, a burn, an incision, a laceration, a penetration wound, a puncture wound, a sore, or an ulcer.

30. The method of alternative 29, wherein the ulcer is a diabetic ulcer, a venous ulcer, a chronic ulcer, or a pressure ulcer.

31. The method of any one of alternatives 25-30, wherein treating or ameliorating the wound promotes full thickness or partial thickness repair, accelerates wound healing, promotes wound closure, causes wound regression, increases epithelialization, increases epithelial layer thickness, increases granulation, increases granular layer thickness, increases collagen deposition, increases capillarization, enhances vascularization, enhances immune modulation, enhances cell migration, or enhances cell growth, or any combination thereof.

32. The method of any one of alternatives 25-31, wherein the composition is applied to a wound area of the subject in an amount of 0.01, 0.05, 0.1, 0.5, 1, 1.5, 2, 2.5, 5, 10, 25, 50, 100, 500 mg, or 1000 mg/cm² or within a range defined by any two of the aforementioned amounts.

33. The method of any one of alternatives 25-32, wherein the composition is applied to the wound at least once daily, once weekly, once monthly, or once yearly.

34. The method of any one of alternatives 25-33, wherein the composition is applied to the wound at least three times a day.

35. The method of any one of alternatives 25-34, wherein the composition is applied to the wound sequentially in 2, 3, 4, 5, 6, 7, 8, 9, 10, or more applications.

36. The method of any one of alternatives 25-35, wherein the composition or system is applied to the wound for a period of 1, 2, 3, 4, 5, 6, 7, 8, 9, 10, 15, 20, 30 or more days, 1, 2, 3, 4, 5, 6, 7, 8, 9, 10, 11, 12 or more months, or 1, 2, 3, 4, 5, 10, or more years.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts a trichrome staining of a mouse skin section showing healing of the skin layer and depicting amelioration of the regular structure of the skin after wound epithelial closure and formation of a granular layer.

FIG. 2 depicts trichrome staining of normal mouse skin section, showing the epithelial layer, the granular layer, and the fat layer.

FIG. 3 depicts trichrome staining of skin section, showing the epithelial layer (small double arrow) and the granular layer (large double arrow) in a treated mouse wound. Collagen deposition is depicted with arrows.

FIG. 4 depicts CD31 staining of a skin section. Capillaries and larger vessels walls are stained in the treated mouse wound.

FIG. 5 depicts CD31 staining of a skin section. Capillaries and larger vessels walls are stained in the treated mouse wound.

FIG. 6A depicts images of visual progression of wound healing for treated and non-wounds of mouse 1. FIG. 6B depicts images of stained microscopic slides taken from mouse 1 tissue after 14 days.

FIG. 7A depicts images of visual progression of wound healing for treated and non-wounds of mouse 2. FIG. 7B depicts images of stained microscopic slides taken from mouse 2 tissue after 14 days.

FIG. 8A depicts images of visual progression of wound healing for treated and non-wounds of mouse 3. FIG. 8B depicts images of stained microscopic slides taken from mouse 3 tissue after 14 days.

FIG. 9A depicts images of visual progression of wound healing for treated and non-wounds of mouse 4. FIG. 9B depicts images of stained microscopic slides taken from mouse 4 tissue after 14 days.

FIG. 10A depicts images of visual progression of wound healing for treated and non-wounds of mouse 5. FIG. 10B depicts images of stained microscopic slides taken from mouse 5 tissue after 14 days.

FIG. 11A depicts images of visual progression of wound healing for treated and non-wounds of mouse 6. FIG. 11B depicts images of stained microscopic slides taken from mouse 6 tissue after 14 days.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Wounds affect millions of subjects, and the costs of wound healing in health care is enormous. Provided herein are compositions and methods for treating or ameliorating a wound, wherein the composition comprises an extracellular matrix (ECM) or ECM protein produced and isolated from small mobile stem (SMS) cells, wherein the ECM or ECM protein may comprise at least one induced cross-link. The compositions, systems, and methods provided herein are generated from SMS cells, thereby providing a reliable and efficient source of ECM and ECM protein production.

Unless defined otherwise, technical and scientific terms used herein have the same meaning as commonly understood by one of ordinary skill in the art to which the present disclosure belongs, See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In some embodiments, the “purity” of any given agent (e.g., antibody, polypeptide binding agent) in a composition may be specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

As used herein, the terms “function” and “functional” and the like refer to a biological, enzymatic, or therapeutic function.

The term “isolated” is meant material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The practice of the present disclosure will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al, Molecular Cloning: A Laboratory Manual (3^{rd} Edition, 2000); DNA Cloning: A Practical Approach, vol. 1 & 11 (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Flames & S. Higgins, eds., 1984); Animal Cell Culture (R. Freshney, ed., 1986); Freshney, R. I. (2005) Culture of Animal Cells, a Manual of Basic Technique, 5^{th} Ed. Hoboken N.J., John Wiley & Sons; B. Perbal, A Practical Guide to Molecular Cloning (3^{rd} Edition 2010); Farrell, R., RNA Methodologies: A Laboratory Guide for Isolation and Characterization (3^{rd} Edition 2005).

### Compositions

Embodiments disclosed herein relate to compositions comprising an ECM or ECM protein isolated from a culture of SMS cells, which may or may not comprise one or more induced cross-links, which have been induced by exposure to a cross-linking agent or a cross-linking technique e.g., radiation, chemical, mechanical, or temperature and the use of said ECM or ECM protein as a medicament, e.g., for facilitating the healing of a wound of a subject such as a chronic wound.

“SMS cells” as used herein refers to a cell or a cell population characterized in that the cells are adherent cells of about 5 μm in diameter. The SMS cells are equi-dimensional, with strict radial symmetry, and exhibit a translucent cytoplasm and circular nucleus that includes a centrally located circle of a different light contrast, as viewed in a light microscope. In addition, SMS cells demonstrate an extraordinary resistance to various non-physiological conditions, including low and high temperature, freezing and thawing in standard growth medium, dehydration, high pH values, and variations of ionic strength. SMS cells are also characterized by their high mobility of up to about 1.5 μm/sec.

As used herein, “extracellular matrix (ECM)” is an extracellular component consisting of an intricate network of ECM proteins and polysaccharides that are secreted by cells. SMS-derived ECM and ECM proteins refers to ECM and ECM protein that has been produced by, derived from, isolated from, or otherwise obtained from a culture of SMS cells. In some embodiments, the ECM or ECM protein isolated from a culture of SMS cells comprises agrin, filaggrin, mucin, secreted phosphoprotein 24 (bone matrix), nidogen, cadherins, clathrin, collagen, defensin, elastin, entactin, fibrillin, fibronectin, vitronectin, keratin, laminin, microtubule-actin cross-linking factor 1, SPARC-like protein, nesprin (nesprin-1, nesprin-2, nesprin-3), fibrous sheath-interacting protein, myomesin, nebulin, keratinocyte proline rich protein, plakophilin, integrin, talins, exportins, transportin, tenascin, pedecan, sortilin-related receptor, tensin, titin, total protein, or a fragment of any one or more of the aforementioned proteins.

In some embodiments, the ECM or ECM protein isolated from a culture of SMS cells comprises at least one induced cross-link, which has been induced by exposure to a cross-linking agent or a cross-linking technique e.g., radiation, chemical, mechanical, or temperature. As used herein, a cross-linked ECM or ECM protein is an ECM or ECM protein that has been modified such that individual polymer chains are bonded to or cross-linked to other polymer chains. Cross-linking may be generated or induced by methods using, for example, radiation, chemical cross-linking, or physical cross-linking. For example, radiation cross-linking may be induced by gamma radiation, ultra violet light, or electron beam radiation (e-beam radiation). Chemical cross-linking may be induced by a cross-linking agent, for example, an acid, a base, a phenol resin, urea resin, melamine resin, epoxy resin, polyurethane, vinylester resin, cross-linked polystyrene, cross-linked rubber, acrylamide polymer, or other chemical cross-linking agents. Physical cross-linking may be induced by a physical cross-linking environment, such as by agitation or shearing force. In some embodiments, the composition comprises a cross-linked ECM or ECM protein, wherein the cross-linked ECM or ECM protein comprises at least one, at least two, at least three, at least four, at least five, at least 10, at least 20, at least 30, or more cross-links.

In some embodiments, the cross-linked ECM or ECM protein is denatured or modified. For example, a cross-linked ECM or ECM protein may be modified with an acetylation, acylation, carboxylation, glycosylation, hydroxylation, lipidation, methylation, pegylation, phosphorylation, prenylation, sulfation, or ubiquitination, or combination thereof.

In some embodiments, the composition further comprises collagen, polygalactin mesh, polylactic-glycolic acid, polycaprolactone, polypyrrole, hyaluronan, chitosan, or xenogenic tissue.

In some embodiments, the composition comprises a cross-linked ECM or ECM protein isolated from a culture of SMS cells, and at least one additional compound. In some embodiments, the at least one additional compound is an antimicrobial agent, a cell, an antibiotic, an anti-inflammatory compound, an analgesic, a growth factor, a carrier, a diluent, an excipient, fragrance, emollient, fatty acid, or combination thereof. In some embodiments, the at least one additional compound is not natively present in said cross-linked ECM or ECM protein.

In some embodiments, the at least one additional compound is an antimicrobial agent. As used herein, an antimicrobial agent is an agent that exhibits antimicrobial activity, or a compound that slows or stops growth or proliferation, slows or stops the rate of growth and/or proliferation, or stuns, inactivates, or kills a miocroorganisms. Antimicrobial agents can encompass the terms antibiotics, antibacterials (bactericidal or bacteriostatic agents), antivirals (virucidal agents), antifungals (fungicidal or fungistatic agents), yeast-inhibiting agents, mold-inhibiting agents, anthelminthics (vermifuge or vermicidal agents), or antiparasitics. In some embodiments, the antimicrobial agent is an antimicrobial peptide, for example, a member of the RNAse A super family, a defensin, cathelicidin, granulysin, histatin, psoriasin, dermicidin, collectin, or hepcidin. In some embodiments, the antimicrobial agent is a C-type lectin family 4, septin 12, or pancreatic ribonuclease.

As used herein, the term “microorganism” includes bacteria, viruses, yeasts, fungi, molds, or protozoa. In some embodiments, microorganisms include superbugs, which are microorganisms that have developed a tolerance for or resistance to disinfecting agents, and are therefore not affected by anti-microbial treatments. In some embodiments, superbugs include, for example, Staphylococcus aureus (including methicillin-resistant S. aureus (MRSA) and vancomycin-resistant S. aureus (VRSA)), extended spectrum beta-lactamase (ESBL), Pseudomonas aeruginosa, Clostridium difficile, Salmonella, Mycobacterium tuberculosis, Escherichia coli (including Carbapenem resistant E. coli), multidrug-resistant Acinetobacter baumannii (MRAB), vancymycin-resistant Enterococcus (VRE), Carbapenem resistant Klebsiella pneumoniae, HIV, hepatitis, or influenza.

In some embodiments, the at least on additional compound is a cell. In some embodiments, the cell is a fibroblast, endothelial cell, keratinocyte, melanocyte, or stem cell, or combination thereof.

In some embodiments, the at least one additional compound is an antibiotic. As used herein, antibiotic refers to a substance that is used to treat or prevent bacterial infection by killing bacteria, inhibiting the growth of bacteria, or reducing the viability of bacteria. Accordingly, the term antibiotic includes, but is not limited to, aminoglycosides (gentamicin, streptomycin, kanamycin), β-lactams (penicillins, cephalosporins, monobactams, and carbapenems), vancomycins, bacitracins, macrolides (erythromycins), lincosamides (clindomycin), chloramphenicols, tetracyclines, amphotericins, cefazolins, clindamycins, mupirocins, sulfonamides and trimethoprim, rifampicins, metronidazoles, quinolones, novobiocins, polymyxins, or gramicidins, or any salts or variants thereof. The antibiotic used in compositions and methods described herein will depend on the type of bacterial infection.

In some embodiments, the at least one additional compound is an anti-inflammatory compound. As used herein, an anti-inflammatory compound refers to compounds for the treatment of inflammation. Anti-inflammatory compounds include, for example, non-steroidal anti-inflammatory drugs (NSAIDs; such as aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (prednisone, betamethasone, budesonide, cortisone, dexamethasone, hydrocortisone, methylprednisolone, prednisolone, trimcinolone, and fluticasone), rapamycin, high density lipoproteins (HDL) and HDL-cholesterol elevating compounds (rosiglitazone), rho-kinase inhibitors, anti-malarial agents (hydroxychloroquine and chloroquine), acetaminophen, glucocorticoids, steroids, beta-agonists, anticholinergic agents, methyl xanthines, gold injections (sodium aurothiomalate), sulphasalazine, penicillamine, anti-angiogenic agents, dapsone, psoralens, anti-viral agents, or statins.

In some embodiments, the at least one additional compound is an analgesic. As used herein, an analgesic is an agent that lessens, alleviates, reduces, relieves, or extinguishes pain in an area of a subject's body (an analgesic has the ability to reduce or eliminate pain or the perception of pain without a loss of consciousness). Analgesics include, for example, opioid analgesics (codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene or pentazocine) or non-opiate analgesics (NSAIDs such as salicylates (aspirin, methyl salicylate, and diflunisal); arylalkanoic acids (indomethacin, sulindac, diclofenac, or tolmetin); N-arylanthranilic acids (fenamic acids, mefenamic acid, or mecflofenarnate); oxicams (piroxicam and meloxicam); coxitis (celecoxib, rofecoxib, valdecoxib, parecoxib, or etoricoxib); sulphonanilides (nimesulide) naphthylalkanones (nabumetone); anthranilic acids (pyrazolidinediones and phenylbutazone); proprionic acids (fenoprofen, flurbiprofen, ibuprofen, ketoprofen, naproxen, or oxaprozin); pyranocarboxylic acids (etodolac); pyrrolizine carboxylic acids (ketorolac); or carboxylic acids.

In some embodiments, the at least one additional compound is a growth factor. As used herein, growth factor refers to an extracellular polypeptide-signaling molecule that stimulates a cell to grow or proliferate. Growth factors include those to which a broad range of cell types responds. Examples of neurotrophic growth factors include, but are not limited to, fibroblast growth factor family members such as basic fibroblast growth factors (bFGF), acidic fibroblast growth factors (aFGF), hst/Kfgf gene products, FGF-3, FGF-4, FGF-6, keratinocyte growth factors (KGF), androgen-induced growth factors (AIGF), ciliary neurotrophic factors (CNTF), nerve growth factors (NGF), brain derived neurotrophic factors (BDNF), Neurotrophin 3 (NT3), Neurotrophin 4 (NT4), bone morphogenic proteins (BMP), glial-cell line derived neurotrophic factors (GDNF), osteoclast stimulating factors, activity-dependent neurotrophic factors (ADNF), cytokine leukemia inhibiting factors (LIF), oncostatin M, calmodulin like proteins, thymosins, insulin-like growth factor binding proteins, epidermal growth factors (EGF), a platelet derived growth factor (PDGF), transforming growth factors (TGF-α and TGF-β 1, 2, & 3), vascular endothelial growth factors (VEGF), hepatocyte growth factors (HGF), erythropoietin (EPO), insulin-like growth factors (IGF-I and IGF-II), interleukin cytokines (IL-1α, IL-1β, IL-2, IL-3, IL-4, IL-5, IL-6, IL-7, IL-8, IL-9, IL-10, IL-11, IL-12, IL-13), interferons (IFN-α, IFN-β, and IFN-γ), tumor necrosis factors (TNFα and TNF-β), or colony stimulating factors (GM-CSF and M-CSF), or a combination thereof.

In some embodiments, the at least one additional compound is a carrier. As used herein, a “carrier” refers to a compound that facilitates the incorporation of a compound into cells or tissues. Exemplary carriers include, but are not limited to, water, saline, buffered saline, dextrose, glycerol, ethanol, partial glyceride mixtures of saturated or unsaturated vegetable fatty acids, waxes, polyethylene-polyoxypropylene-block polymers, starches such as corn starch or potato starch, or combinations thereof.

In some embodiments, the at least one additional compound is a diluent. As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

In some embodiments, the at least one additional compound is an excipient. As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

In some embodiments, the at least one additional compound is a fragrance. As used herein, “fragrance” refers to any perfume, odor-eliminator, odor masking agent, the like, and combinations thereof. In some embodiments, a fragrance is any substance which may have an effect on a consumer, or user's, olfactory senses.

In some embodiments, the at least one additional compound is an emollient. As used herein, “emollient” refers to a material useful for the prevention or relief of dryness, as well as for the protection of the skin. Wide varieties of suitable emollients are known and may he used herein. See, e.g., Sagarin, Cosmetics, Science and Technology, 2nd Edition, Vol. 1, pp. 3243 (1972), which contains numerous examples of materials suitable as an emollient and is fully incorporated herein by reference.

In some embodiments, the at least one additional compound is a lipid, including a fat, a fatty acid, or a derivative of fatty acids. Lipids may include natural oils, waxes, or steroids, and may be used depending on the route of administration or type of formulation.

The presence, amount, quantity, and proportion of the at least one additional compound may depend upon the formulation of the composition, whether for topical, oral, intravenous, or other formulation type. The presence, amount, quantity, and proportion of the at least one additional compound may depend on the type of wound, the severity of the wound, the severity of pain, infection, or inflammation, or the intended treatment. Thus, the amount of at least one additional compound may be, for example, an amount ranging from 0.001% to 90%, such as 0.001, 0.005, 0.01, 0.05, 0.1, 0.5, 1, 2, 3, 4, 5, 10, 15, 20, 25, 30, 35, 40, 50, 60, 70, 80, or 90 percent, or an amount within a range defined by any two of the aforementioned values.

In some embodiments, the compositions provided herein are formulated for various modes of administration, including for example, for topical or localized administration. In some embodiments, the methods of the present disclosure further contemplate systemically or parenterally, such as subcutaneously, intraperitoneally, intravenously, intraarterially, orally, enterically, subdermally, transdermally, sublingually, transbuccally, rectally, or vaginally. In some embodiments, the compositions are formulated as an implant for implantation into a subject.

In some embodiments, depending on the mode of administration, the composition is formulated as an implant, a powder, an aerosol, a cream, an emulsion, a foam, a foamable liquid, a gel, a lotion, an ointment, a paste, a salve, a serum, a solution, or a spray. In some embodiments, the composition is formulated as a therapeutic composition or as a cosmetic composition.

Some embodiments provided herein relate to a system for wound healing. In some embodiments, a system for wound healing comprises a composition as described herein and a wound dressing material, formulated as a system for wound healing. In some embodiments, the wound dressing material is a bandage, a wipe, a gauze, a sponge, a mesh, a pad, an adhesive bandage, a nylon, or an absorbent wound dressing material. In some embodiments, the composition is present in the system an amount of 0.005% vol/w % to 90% vol/w %, such as 0.005, 0,006, 0.007, 0,008, 0.009, 0.01, 0.02, 0.03, 0.04, 0.05, 0.06, 0.07, 0.08, 0.09, 0.1, 0.2, 0.3, 0.4, 0.5, 0.6, 0,7, 0,8, 0.9, 1, 1.1, 1.2, 1.3, 1.4, 1.5, 1.6, 1.7 1.8, 1.9, 2, 3, 4, 5, 6, 7, 8, 9, 10, 15, 20, 35, 40, 45, 50, 60, 70, 80, or 90% volume of composition per weight of wound dressing material (vol/w %), or greater or within a range defined by any two of the aforementioned amounts.

In some embodiments, the composition may be configured for incorporation into a wound dressing material, including a bandage, a wipe, a gauze, a sponge, a mesh, a pad, an adhesive bandage, a nylon, an absorbent wound dressing material, or other wound dressing material. The composition may be used to saturate, impregnate, cover, coat, or otherwise be incorporated into a wound dressing material.

In some embodiments, compositions comprising a cross-linked ECM or ECM protein isolated from a culture of SMS cells are added, incorporated, coated on, applied to, saturated with, impregnated with, covered with, or otherwise incorporated or contacted with a wound dressing material in an amount of 0.005% to 90% volume composition to weight wound dressing material (vol/w %), such as 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7. 1.8, 1.9, 2, 3, 4, 5, 6, 7, 8, 9, 10, 15, 20, 35, 40, 45, 50, 60, 70, 80, or 90% volume of composition per weight wound dressing material (vol/w %), or greater, or an amount within a range defined by any two of the aforementioned values. In some embodiments, the composition is contacted with a wound dressing in an amount of 0.05 μg per mg wound dressing material to 1 mg per mg wound dressing material, such as 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, 300, 350, 400, 450, 500, 600, 700, 800, 900, or 1000 μg/mg, or an amount within a range defined by any two of the aforementioned values. Thus, in some embodiments, the composition including an ECM or ECM protein isolated from a culture of SMS cells, preferably comprising at least one induced cross-link, which has been induced by exposure to a cross-linking agent or a cross-linking technique e.g., radiation, chemical, mechanical, or temperature, is incorporated into or onto a wound dressing material, for example, to saturate, impregnate, cover, coat, or otherwise be incorporated into the wound dressing material. Coating can include, for example, dip coating or surface modifying a material with a composition comprising an ECM or ECM protein isolated from a culture of SMS cells, preferably comprising at least one induced cross-link, which has been induced by exposure to a cross-linking agent or a cross-linking technique e.g, radiation, chemical, mechanical, or temperature.

In some embodiments, the composition for incorporation into a wound dressing material comprises a plurality of different ECM proteins and a plurality of polysaccharides, such as agrin, filaggrin, secreted phosphoprotein 24 (bone matrix), vitronectin, mucin, nidogen, cadherins, clathrin, collagen, defensin, elastin, entactin, fibrillin fibronectin, keratin, laminin, microtubule-actin cross-linking factor 1, SPARC-like protein, nesprin (nesprin-1, nesprin-2, nesprin-3), fibrous sheath-interacting protein, myomesin, nebulin, plakophilin, integrin, talins, exportins, transportin, keratinocyte prolific rich protein, tenascin, perlecan, sortilin-related receptor, tensin, titin, total protein, hyaluronic acid, cellulose, or a fragment, analogue, or derivative of any one or more of the aforementioned proteins and polysaccharides.

### Methods of Making Compositions and Systems

Some embodiments provided herein relate to methods of making the compositions provided herein. In some embodiments, the methods comprise culturing a population of small mobile stein (SMS) cells for a time sufficient to form an extracellular matrix (ECM) or ECM protein, which adheres to a culture vessel or is present in the culture media e.g. floating or non-adherent to the culture vessel. In some embodiments, the methods further comprise removing the ECM or ECM protein from the culture vessel or media to obtain an isolated ECM or ECM protein. In some embodiments, the methods further comprise, washing the isolated ECM or ECM protein. In some embodiments, the methods further comprise drying the ECM or ECM protein, for instance into a powder. In some embodiments, the methods further comprise introducing at least one cross-link into said ECM or ECM protein such as by exposure to a radiation, preferably gamma radiation, ultra violet light radiation, x-ray radiation, or electron beam radiation (e-beam radiation). In some embodiments, the methods further comprise introducing at least one cross-link into said ECM or ECM protein by exposure to a chemical, or a physical cross-linking environment, such as an acid, base, or a shearing force. In some embodiments, the methods further comprise freezing the ECM or ECM protein. In some embodiments, the methods further comprise joining the ECM or ECM protein to a support. In some embodiments, the support comprises a cellulose or a sugar or a sugar alcohol.

“Cell culture” or “cultured cell”, as used herein, refer to cells or tissues that are maintained, cultured, cultivated or grown in an artificial, in vitro environment. Included within this term are continuous cell lines (e.g. with an immortal phenotype), primary cell cultures, finite cell lines (e.g., non-transformed cells), and any other cell population maintained in vitro. In this connection, a primary cell is a cell that is directly obtained from a tissue or organ of an animal, including a human, in the absence of culture. Typically, though not necessarily, a primary cell is capable of undergoing ten or fewer passages in vitro before senescence or cessation of proliferation.

“Maintenance” means continued survival of a cell or population of cells, at times, with or without an increase in the numbers of cells. “Proliferation”, “propagation”, “expansion” and “growth”, which may be used interchangeably with each other, refer to an increase in cell number. According to one embodiment, this term refers to a continuous survival of the cells for at least 6, 7, 8, 9, 10, 11, 12, 13, 14, 15, 16, 17, 18, 19, 20, 21, 22, 23, 24, 25, 26, 32, 48, 52, 104 or more weeks or within a range defined by any two of the aforementioned time frames. In the alternative, the continuous survival of the cells continues for at least 25, 26, 27, 28, 29, or 30 or more passages or within a range defined by any two of the aforementioned number of passages.

“Cell suspension” as used herein, refers to a culture of cells in which the majority of the cells freely float in the medium, typically a culture medium (system), and the cells floating as single cells, as cell clusters and/or as cell aggregates. In other words, the cells survive and propagate in the medium without being attached to a solid or semi solid substrate. “Adherent cells” as used herein refers to a cell or cell population that adheres to a substrate or surface.

“Culture system” as used herein, refers to culture conditions for supporting the maintenance and propagation of SMS cells or somatic cells derived therefrom, as well as, under selected conditions, for supporting derivation and propagation of undifferentiated or differentiated SMS cells. The term denotes a combination of elements, which can include a basic medium (a cell culture medium usually including a defined base solution, which includes salts, sugars and amino acids) and a serum replacement supplement. The culture system may further include other elements such as, without being limited thereto, an extracellular matrix (ECM) component, additional serum or serum replacements, a culture (nutrient) medium and other exogenously added factors, which together provide suitable conditions that support SMS cell growth, cell culture maintenance, cell differentiation, or expression of various molecules. In the relevant context, the term “culture system” also encompasses the cells cultured therein.

SMS cells may be cultured in T25 flasks using growth medium (e.g., in an incubator at 37° C. and 5% CO₂). The SMS cell population may contain a heterogeneous cell population of undifferentiated SMS cells and SMS derived differentiated cells. SMS undifferentiated cells are present as a floating and an adherent fraction and the floating fraction is predominantly undifferentiated SMS cells (e.g., greater than or equal to 50%, 55%, 60%, 65%, 70%, 75%, 80%, 85%, 90%, or 95% undifferentiated SMS cells or an amount of undifferentiated SMS cells that is within a range defined by any two of the aforementioned values). Accordingly, some alternatives concern a suspension of non-adherent and undifferentiated SMS cells, which are preferably grown in a liquid media in a manner that prevents adherence (e.g., in a polypropylene vessel or flask). In some alternatives, the floating and undifferentiated SMS cell population is greater than or equal to 50%, 55%, 60%, 65%, 70%, 75%, 80%, 85%, 90%, or 95% undifferentiated SMS cells or an amount of undifferentiated SMS cells that is within a range defined by any two of the aforementioned values.

Undifferentiated SMS cells can be isolated or purified by differential centrifugation, removing clumps of cells or differentiated cells at low centrifugation speed followed by centrifuging undifferentiated SMS cells at high speed. Alternatively, the undifferentiated cells may be isolated by filtration, including differential filtration using filters having progressively smaller pore sizes to a pore size of 3-5 μm. Alternatively, the undifferentiated cells may be isolated by immune conjugation (e.g., a binding partner specific for a stem cell receptor on SMS cells, wherein the binding partner, such as an antibody, is conjugated to a bead, such as a magnetic bead or via FACS cell sorting), or differential filtration using filters having progressively smaller pore sizes to a pore size of 3-5 μm The isolated undifferentiated SMS cells are examined under the microscope for homogeneity. By passing the cells for at least 25 passages (e.g., 25, 26, 27, 28, 29, 30, 31, 32, 33, 34, 35, 36, 37, 38, 39, or 40 passages or within a range defined by any two of the aforementioned number of passages) prior to employing one or more of the isolation protocols above, one can obtain an improved homogeneous population of SMS cells.

The SMS cells can be grown a variety of mediums with or without serum and with or without inclusion of a differentiation induction compound (e.g., insulin). The cell growth medium is replaced every week by centrifuging the SMS cells at 4200 g for 15 min. The centrifugation may be varied at 3000 g, 3500 g, 4000 g, 4100 g, 4200 g, 4300 g, 4500 g, or 5000 g or by centrifugation at a speed that is within a range defined by any two of the aforementioned speeds, and the time adjusted accordingly. Under these conditions, the volume size of the medium (cell crowdedness) is growth limiting to SMS cells. The SMS cell homogeneity is assessed microscopically and the SMS cell count is estimated by assessing spectroscopically turbidity of the suspension and/or by measuring the size of the pellet after centrifugation at high speed. Suspension cultures of SMS cells facilitate transfer and cloning as the cells are easily split and/or transferred to a new tube with growth medium and such methods of splitting, and/or transferring a culture of cells from an existing culture to a new culture is performed without using an enzyme that liberates the cells from the culture dish or a basal cell layer (e.g., trypsin). SMS cells predominantly grow as individual cells, without clumping and the SMS cell suspensions can remain undifferentiated despite transfer procedures. The suspension culture is also scalable such that increasing the volume of the medium increases the number of cells obtained.

The SMS cells are cultured to generate ECM or ECM proteins. In some alternatives, the SMS cells are transformed with a gene encoding an ECM protein. SMS cells can be grown in suspension (whether transformed with a gene encoding an ECM protein or a native SMS cell) and are preferably maintained in an undifferentiated state. Several passages of the cells are made so as to obtain a homogeneous population of undifferentiated SMS cells in suspension (e.g., greater than or equal to 50%, 55%, 60%, 65%, 70%, 75%, 80%, 85%, 90%, or 95% undifferentiated SMS cells or an amount of undifferentiated SMS cells that is within a range defined by any two of the aforementioned values). Chemical inducers of ECM or ECM protein production are provided to the culture (e.g., a hedgehog inhibitor and/or a TGF/BMP activator) and the ECM or ECM protein generated in the suspension is recovered (e.g., by filtration, centrifugation, or immune conjugation). Similarly, ECM or ECM protein can be made with adherent SMS cells. In this alternative, SMS cells (whether transformed with an gene encoding an ECM protein or a native SMS cell) are seeded on T25 flasks or plates (e.g., polystyrene; physical surface inducers) and the adherent cells are grown for several passages so as to obtain a homogeneous population of undifferentiated SMS cells in suspension (e.g., greater than or equal to 50%, 55%, 60%, 65%, 70%, 75%, 80%, 85%, 90%, or 95% undifferentiated SMS cells or an amount of undifferentiated SMS cells that is within a range defined by any two of the aforementioned values). Once a desired level of homogeneity is obtained, chemical inducers of ECM or ECM protein production are provided to the medium, including, for example, a hedgehog inhibitor and/or a TGF/BMP activator. The ECM generated in the suspension is then recovered or isolated (e.g., by filtration, centrifugation, or immune conjugation). After about two weeks, floating ECM is preferably harvested by centrifugation at high speed. Adherent ECM or ECM protein is harvested at about the same time by scrapping the proteins from bottom of the flask or dish. Cross-linking of said ECM or said ECM protein can be induced by several approaches e.g., by exposure to a cross-linking agent or a cross-linking technique such as radiation, chemical, mechanical, or temperature, as described above.

In some embodiments, a SMS-derived ECM or ECM protein produces one or more anti-microbial compounds or the ECM or ECM protein itself has anti-microbial properties, such as antibacterial, antiviral, or antifungal properties. In some embodiments, the antimicrobial compounds or ECM or ECM proteins having antimicrobial properties produced by SMS cells comprise dermicidin, collectin, C-type lectin family 4, septin 12, defensin, or pancreatic ribonuclease. In some embodiments, SMS cells may be grown in the absence of an antibiotic due to the self-production of anti-microbial compounds.

In some alternatives, the ECM or ECM protein is also decellularized (e.g., by using a chemical, physical, and/or enzymatic approach). Preferably, a decellularization approach is configured such that the ECM or ECM protein scaffold maintains its structural and chemical integrity. In addition, various molecular components of the SMS derived ECM or ECM protein can be enriched or isolated. The ECM or ECM protein produced by the aforementioned SMS cells, whether cross-linked or not, may be freeze dried into powder and stored as such. SMS-derived ECM or ECM protein in powder or other form is contemplated for use in the compositions and systems described herein.

In some alternatives, ECM or ECM protein is produced or isolated from SMS cells growing on a scaffold. SMS cells are cultured on various scaffolds (e.g., native decellularized bone, soft decellularized collagen, a carbon support, such as an activated carbon, carbon black, a carbon film, a carbon cloth, nanotube, or microtube or a medical device or implant composed of carbon such as a stent or shunt) in a suitable growth medium (e.g., DMEM with or without serum). In some alternatives, the support is free-floating in the growth medium. Differentiation induction compounds may be added. Microscopic observation indicates that the SMS cells change shape drastically after differentiation, and the SMS cell differentiation varies depending on the nature of the scaffold and/or the type of differentiation compound added. SMS cell attachment, growth, and differentiation is influenced by varying the medium. SMS cells and cells derived from it produce extracellular matrix and tissue like structures attached to the scaffolds.

In some embodiments, the ECM or ECM protein derived from SMS cells is combined with at least one additional component to formulate a composition. For example, the ECM or ECM protein derived from SMS cells, which can be cross-linked, can be combined with one or more antimicrobial agents, cells, antibiotics, anti-inflammatory compounds, analgesic, growth factors, carriers, diluents, excipients, fragrances, emollients, fatty acids, or combination thereof for formulation of a composition.

In some embodiments, the ECM or ECM protein derived from SMS cells is formulated into a powder, an aerosol, a cream, an emulsion, a foam, a foamable liquid, a gel, a lotion, an ointment, a paste, a salve, a serum, a solution, or a spray. In some embodiments, the composition is used in a system, wherein the system includes the composition and a wound dressing material. Thus, the composition may be configured for incorporation into a wound dressing material, including a bandage, a wipe, a gauze, a sponge, a mesh, a pad, an adhesive bandage, a nylon, an absorbent wound dressing material, or other wound dressing material. The composition may be used to saturate, impregnate, cover, coat, or otherwise be incorporated into a wound dressing material.

### Method of Using the Compositions

Some embodiments provided herein relate to a method of treating or ameliorating a wound comprising contacting a wound with a composition described herein or with a system described herein.

As used herein, the terms “treating,” “treatment,” “therapeutic,” or “therapy” do not necessarily mean total cure or abolition of a wound. Any alleviation of any undesired signs or symptoms of a wound, to any extent can be considered treatment and/or therapy, Treatment may include enhancing, improving, accelerating, or ameliorating wound healing.

Treatment may include administration of a wound care composition alone or a wound dressing material that has a wound care composition incorporated therein. When used alone, the wound care composition may he administered topically, orally, subcutaneously, as an implant, or in other means in order to properly treat the wound. When the composition is incorporated into a wound dressing material as a system, the wound dressing material is applied to the wound to treat the wound, or is used as an implant to treat the wound.

The term “wound” is understood to mean a break in the continuity of body tissues with or without substance loss, such a break in general being caused by mechanical injuries or physically caused cell damage. A wound can include skin damage, a burn, radiation damage, sunburn, an abrasion, a laceration, an incision, a sore, a puncture wound, a penetration wound, a gunshot wound, or a crushing injury. A wound may include mechanical wounds, including cutting, laceration, scratch, abrasion, piercing, puncture, penetration, crushing, blunt force trauma, and contusion wounds. A wound may include thermal wounds, which are caused by the action of extreme heat or cold. A wound may include chemical wounds caused by the action of chemicals, in particular by erosion by acids or alkalis. A wound may further include tissue breaks or damage that arise under the action of actinic radiation, e.g. ultraviolet radiation and/or ionizing radiation, are described as radiation wounds. A wound may be necrotizing, infected, chronic, or acute. Thus, a wound may be a partial thickness wound or a full thickness wound, a surface wound or an internal wound, or a chronic wound or acute wound. A wound may be skin damage, a burn, an abrasion, a laceration, an incision, a contusion, a lesion, an ulcer, or a sore. Wounds may include diabetic ulcers, venous ulcers, chronic ulcers, or pressure ulcers. Wounds may include severe burns, tumor excision, or trauma. A wound may also include damage to the skin, and can include, for example damage caused by trauma, burn, surgery, or other type of damage.

As used herein, the term “wound healing” described the phases of a wound from injury to healing. In the first phase, also described as latency or inflammatory phase, within the first hours after wounding has occurred, exudation of body fluids takes place, in particular of blood, to free the wound opening from foreign bodies, germs and dead tissue. Next, a scab, which protects the wound externally from the penetration of germs and foreign bodies, is formed through clotting of the blood that has emerged. After the formation of the scab, the resorption phase of the latency phase begins, in which a catabolic autolysis also takes place, which includes macrophage migration into the wound tissue and phagocytosis of coagulated blood in the wound opening. Foreign bodies or microorganisms that may have penetrated are degraded in this phase, which can be associated with mild to moderate inflammatory symptoms. Further, in the resorption phase the build-up of the basal epithelium and of granulation tissue begins. After about one to three days after causation of the wound, the latency phase is generally completed and the latency phase passes into the second phase, a proliferation or granulation phase, which in general lasts from the fourth to the seventh day after the injury. During this phase, anabolic repair begins. This repair includes the formation of collagen by fibroblasts. In the final phase, the repair or epithelization phase, which begins from about the eighth day after the occurrence of the wound, final scar tissue is formed and the squamous epithelium of the skin is renewed. The scar tissue formed has neither sebaceous nor sweat glands and appears white to mother-of-pearl on the skin. In contrast to undamaged tissue, the collagen in the scar tissue is no longer complexly linked, but is instead aligned parallel.

In some embodiments, treating or ameliorating a wound promotes full thickness or partial thickness repair, accelerates wound healing, promotes wound closure, causes wound regression, increases epithelialization, increases epithelial layer thickness, increases granulation, increases granular layer thickness, increases collagen deposition, increases capillarization, enhances vascularization, enhances immune modulation, enhances cell migration, or enhances cell growth, or any combination thereof.

Further information on the term “wound healing” can be found in Pschyrembel—Clinical Dictionary, 257^{th} edition, 1994, Verlag de Gruyter, Berlin/New York, which is expressly incorporated by reference herein in its entirety.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. A subject in need includes a subject that is suffering from a wound, including a partial thickness wound, a full thickness wound, a surface wound, an internal wound, a chronic wound, an acute wound, skin damage, a burn, an abrasion, a laceration, an incision, a contusion, a lesion, an ulcer, a diabetic ulcer, a venous ulcer, a chronic ulcer, a pressure ulcer, or a sore.

In some embodiments, the composition is applied to a wound area in an amount and for a period sufficient to treat and/or ameliorate the wound. As can be appreciated, wounds may vary significantly in severity, size, ability to heal, or chronicity, and therefore, the amount of composition or the length of time for treatment will vary. However, by way of example, a composition or system may be administered in an amount of 0.01, 0.05, 0.1, 0.5, 1, 1.5, 2, 2.5, 5, 10, 25, 50, 100, 500 mg, or 1000 mg/cm² of wound area, or in an amount within a range defined by any two of the aforementioned values. Also by way of example, the composition or system may be applied to the wound at least three times daily, once daily, once weekly, once monthly, or once yearly, or in a frequency within a range defined by any two of the aforementioned values. Also by way of example, the composition or system may be applied to a wound sequentially in 2, 3, 4, 5, 6, 7, 8, 9, 10, 15, 20, 30, 40, 50 or more applications, or in a number of applications within a range defined by any two of the aforementioned values. Also by way of example, the composition or system may be applied to a wound for a period of 1, 2, 3, 4, 5, 6, 7, 8, 9, 10, 15, 20, 30 or more days, 1, 2, 3, 4, 5, 6, 7, 8, 9, 10, 11, 12 or more months, or 1, 2, 3, 4, 5, 10, or more years, or for a length of time within a range defined by any two of the aforementioned values.

The invention is generally disclosed herein using affirmative language to describe the numerous embodiments. The invention also includes embodiments in which subject matter is excluded, in full or in part, such as substances or materials, method steps and conditions, protocols, or procedures.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1

### Wound Healing Using Cross-Linked ECM Derived from SMS Cells

The following example demonstrates the wound healing effects of cross-linked ECM derived from SMS cells.

### Culture of SMS Cells

SMS cells are cultured, and ECM isolated from the SMS cells as previously described, for example in WO 2017/172638. Briefly, SMS cells are grown in T25 flask using growth medium (37° C. and 5% CO2). The SMS cell population may contain a heterogeneous cell population of undifferentiated SMS cells and SMS derived differentiated cells.

Undifferentiated SMS cells can be isolated by differential centrifugation, removing clumps of cells or differentiated cells at low centrifugation speed followed by centrifuging undifferentiated SMS cells at high speed. Alternatively, the undifferentiated cells may be isolated by filtration, including differential filtration using filters having progressively smaller pore sizes to a pore size of 3-5 μm. The isolated undifferentiated SMS cells are examined under the microscope for homogeneity.

Undifferentiated SMS cells are grown in polypropylene tube (such as the bioreactor tubes: 15 ml provided by the manufacturer Techno Plastic Products AG, TPP).

The following example of growth medium is used: high sugar basal medium (Dulbecco's Modified Eagle Medium (DMEM), [+] 6 g/L D-glucose, [−] sodium pyruvate, [−] L-glutamine, [−] Phenol red), to which 1% GlutaMAX™-I (100×), 10% calf serum, and 5 μg/mL human insulin was added. Alternatively, a medium not containing any calf serum can he used. The cells are suspended occasionally by swirling.

Complete medium is replaced every week by centrifuging the SMS cells at 4200 g for 15 min. The centrifugation may be varied at 3000 g, 3500 g, 4000 g, 4100 g, 4200 g, 4300 g, 4500 g, or 5000 g or by centrifugation at a speed that is within a range defined by any two of the aforementioned speeds, and the time adjusted accordingly.

Under these conditions, the volume size of the medium (cell crowdedness) is growth limiting to SMS cells. The SMS cell homogeneity is assessed microscopically and the SMS cell count is estimated by assessing spectroscopically turbidity of the suspension and/or by measuring the size of the pellet after centrifugation at high speed.

The SMS cell growth potential is assessed by inoculating cells into a. new tube with growth medium. SMS cells grow mainly as individual cells not as clumps and remain under this condition mainly undifferentiated. The suspension culture is scalable such that increasing the volume of the medium increases the number of cells obtained.

Cross-Linked ECM Isolated from a Culture of SMS Cells

The SMS cell culture medium is switched by centrifuging the cells at high speed (for example, at 4200 g for 15min) and suspending in a new growth medium.

SMS cells are seeded on T25 or plates (polystyrene; physical surface inducers) and grown using a growth medium (37° C. and 5% CO2). Chemical inducers of ECM and ECM protein production are provided to the medium, including, for example, a hedgehog inhibitor and a TGF/BMP activator, at growth conditions (37° C. and 5% CO2). The complete medium is added or replaced twice weekly.

After about two weeks, floating ECM or ECM protein is harvested by centrifugation at high speed. Adherent ECM or ECM protein is harvested by scrapping the bottom using a scrapper. Various ECM proteins are produced depending on induction and growth conditions.

ECM or ECM protein is decellularized using various methods known in the art. For example, chemical, physical, and enzymatic methods can be employed to decellularize the ECM or ECM protein, ensuring that the ECM or ECM protein scaffold maintains its structural and chemical integrity. In addition, various molecular components of the SMS derived ECM or ECM protein are enriched or isolated, ECM or ECM protein in various tissues of various organs can be shown to be similar or identical to ECM or ECM protein obtained from in vitro SMS cell culture.

SMS cell produced ECM or ECM protein may be freeze dried into powder and stored as such. ECM or ECM protein in powder or other form are used in wound healing medicaments and therapies.

### Mouse Model of Wound

A diabetic animal model (mouse) having an open full excision skin wound was used to measure the effects of the composition comprising cross-linked ECM isolated from a culture of SMS cells.

The diabetic ulcer represents a large fraction (˜30%) of non-healing wounds of human patients, Rodents, especially the diabetic mouse model (BKS.Cg-Dock7m+/+Leprdb//J mice, stock #000642, Homozygous) has been used frequently in testing compounds for wound healing. The use of splinting prevents skin contraction during the healing of the mouse wound, which makes the model more comparable to the healing process of human skin wounds.

Diabetic mice were purchased from Jackson laboratory. Mice homozygous for the diabetes spontaneous mutation (Leprdb) become identifiably obese around three to four weeks of age. Elevation of plasma insulin begins at 10 to 14 days of age and of blood sugar at four to eight weeks. Homozygous mutant mice are polyphagic, polydipsic, and polyuric. The course of the disease is markedly influenced by genetic background. A number of features are observed on the C57BLKS background, including an uncontrolled rise in blood sugar, severe depletion of the insulin-producing beta-cells of the pancreatic islets, and death by 10 months of age. Exogenous insulin fails to control blood glucose levels and gluconeogenic enzyme activity increases. Peripheral neuropathy and myocardial disease are seen in C57BLKS-Leprdb homozygotes. Wound healing is delayed, and metabolic efficiency is increased. Female homozygotes exhibit decreased uterine and ovarian weights, decreased ovarian hormone production and hypercytolipidemia in follicular granulosa and endometrial epithelial tissue layers (Garris et al., 2004, Garris et al., 2004). The mice were handled, operated, monitored and housed at LaBiomed vivarium facility. For the study, six confirmed diabetic mice were used.

Animals were fasted for at least 12 hours prior to the blood collection. The animals were restrained with one hand, the tail wiped with alcohol and allowed to thy before pricking it. When the skin was dry, the lateral tail vein was visualized and the skin above the vein pricked with a clean 20-22 gauge needle.

The needle was removed and pressure was applied around the needle hole until a small drop of blood was visible. The glucose meter strip was placed next to the drop of blood so it can be sucked into the strip and a measurement was taken. Pressure was applied to the tail above the needle prick with a clean gauze to stop any fluffier blood flow. Each animal was returned to its cage and food provided ad lib. Only diabetic mice exhibiting high glucose levels were selected for the experiment.

Each mouse was inflicted with two wounds, a test wound and a control wound. General anesthesia was induced using 5% isoflurane in 100% oxygen (flow rate 1 L/min) and anesthesia maintained using 1-3% isoflurane. The deep pedal reflexes of the mouse were suppressed and the mouse was placed in the prone position. The operative region was prepared by removing fur with clippers from the base of the neck to 3 cm further down the back and between the two shoulder blades. Wet gauze swabs were used to ensure all cream and remaining fur was removed. The skin was wiped with an alcohol swab and three applications of 10% povidone-iodine (Betadine) and the mouse draped. A sterile 6 mm biopsy punch was used to outline two circular patterns for the wound on either side of the mouse's midline at the level of the shoulders. Serrated forceps were used to lift the skin in the middle of the outline and iris scissors to create a full-thickness wound that extends through the subcutaneous tissue, including the panniculus camosus. The circular piece of tissue was excised. The process was repeated for the wound on the other side of the midline. The plastic protective coating from each side of the silicone splint was removed. Vetbond was applied to one side of a silicone splint. The splint was centered over the wound and anchored with interrupted 6-0 nylon sutures to ensure positioning. The splinting process was repeated on the other wound.

To one wound, 1.5-2 mg of composition was applied evenly on the open wound using a plastic sterile spatula to one wound. Nothing was applied to the control wound. Each wound was covered with two layers of the transparent occlusive dressing Tegaderm.

Meloxicam SR (4 mg/kg) SQ injection was administered for post-operative pain relief, once and repeated every 72 hrs if needed. After the surgery animals were individually caged and maintained on heat mats until fully recovered. The animals were monitored twice daily for manifestations of pain and weight loss. The mice were fed regular chow diet.

The mouse wounds were photographed. Photography at day 0 and day 7 were done after sedating the animal and removing the Tegaderm transparent dressing. Photography at days 3-4 or 10-11 were done while keeping the Tegaderm. Photography at day 14 was done before removing Tegaderm, after euthanasia, and after removing Tegaderm, as well as following removal of splint and excision of the tissue.

The mice were euthanized after 14 days using CO₂. The Tegaderm was removed carefully, the sutures at the splints cut, and splints removed. Two skin tissue full excision samples, covering the whole wound and tissue surrounding it, were taken from each mouse, one for the left side wound another from the right side wound and a third sample representing a non-inflicted wound skin sample.

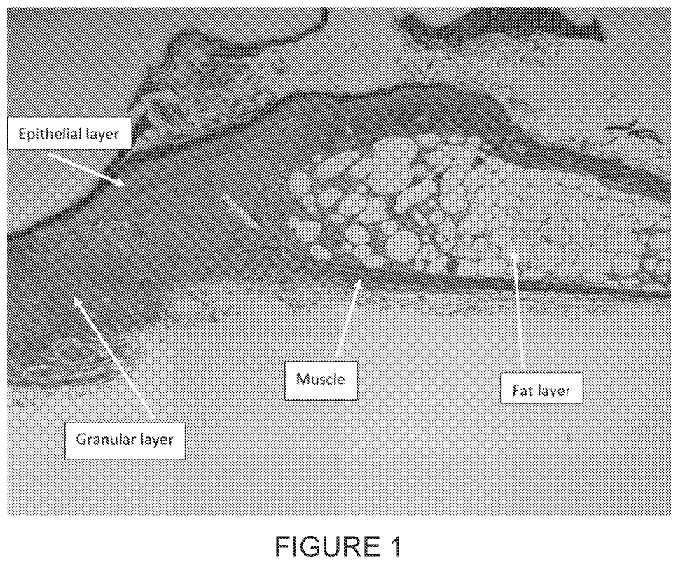

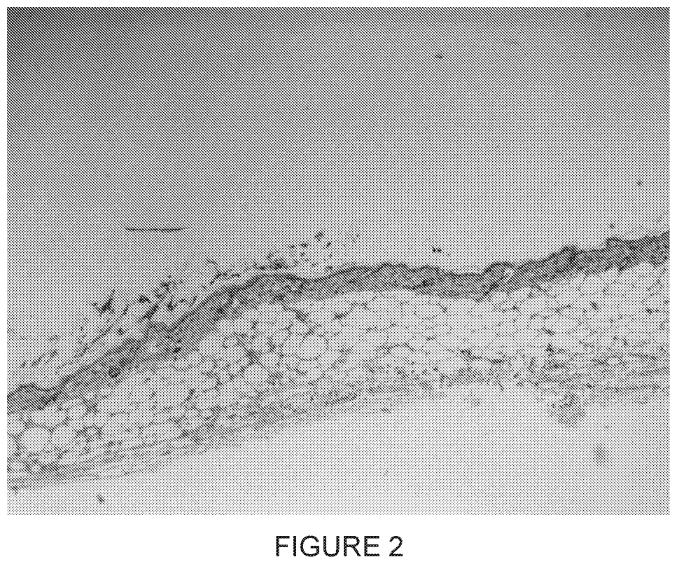

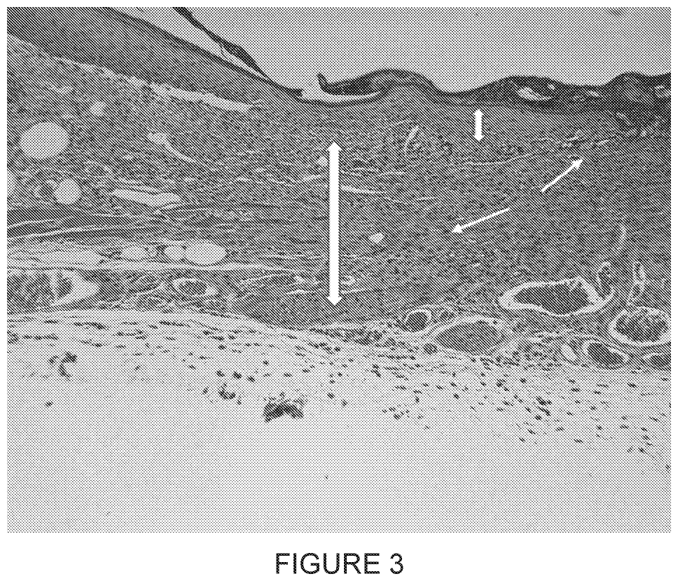

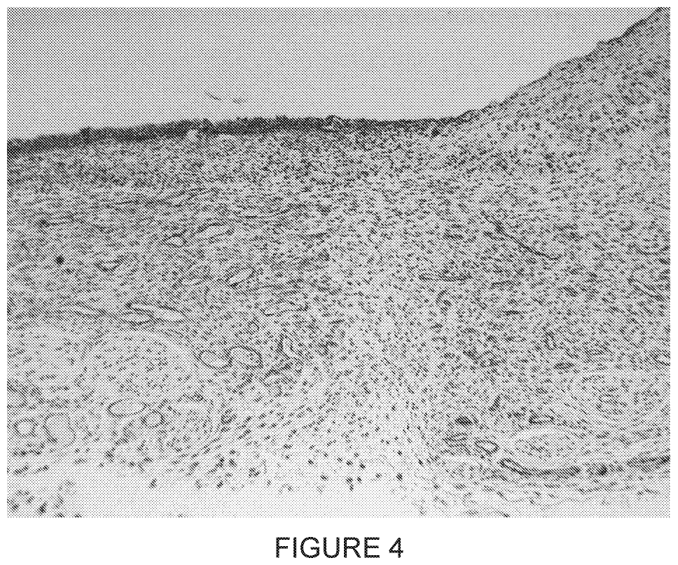

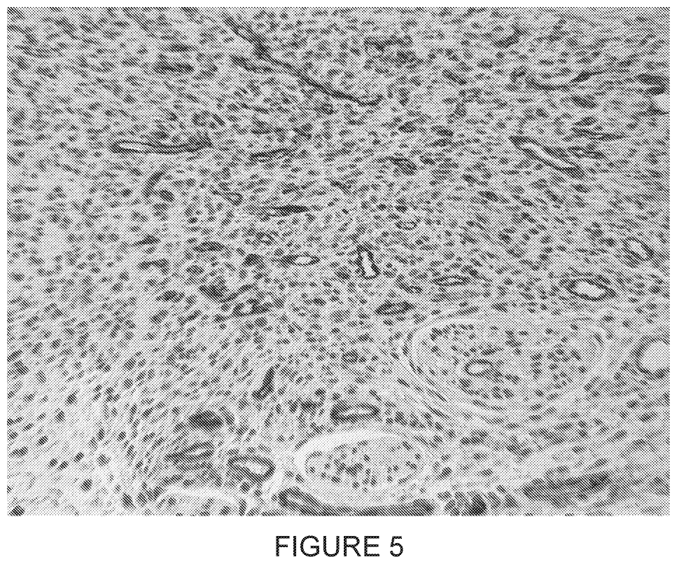

The excised skin tissue was placed and spread on thick paper to prevent contraction of the tissue. The excised wound were photographed and then immersed into a formalin solution. The tissue was removed after 24-48 hours to denatured ethanol and refrigerated until shipment to a histopathology laboratory (IDEXX BioResearch). All tissues were embedded in paraffin and sliced. Slides were made, and tissues were stained using regular Hematoxylin Eosin, Masson Trichrome chemical staining (see FIGS. 1-3) and CD31 immunohistochemical staining (see FIGS. 4-5). CD31 immunohistochemical analysis was performed on paraffin sections using antibodies to CD31. Five-μm paraffin sections were mounted onto charged microscope slides and treated to 55° C. heat on a slide warmer overnight. Sections were de-waxed in xylene, rehydrated through graded concentrations of ethanol, and rinsed in distilled water. Antigen retrieval was performed by immersing sections in heated 10 mM citrate buffer (pH 6.0) for 30 min, and then cooled to room temperature. Sections were treated with 3% hydrogen peroxide, and rinsed prior to incubation in blocking buffer with 5% bovine serum albumin for 20 mM. Sections were then incubated for 60 min at room temperature with anti-CD31 antibody (1:50 dilution; ab28364; Abcam, Inc., Cambridge, Mass.). Sections were washed, incubated with a horseradish peroxidase-labeled polymer conjugated to anti-rabbit antibodies (EnVision®, DAKO Aligent, Carpenteria, Calif.). Bound antibodies were visualized following incubation with a chromogen (NovaRED™, SK-4800, Vector Laboratories, Burlingame, Calif.) for 7-10 minutes. Sections were counterstained with Mayer's hematoxylin, dehydrated, and cover-slipped. The slides were returned for microscopic evaluation.

The primary outcome measures were: (1) Visually inspected wound closure by epithelialization; (2) Microscopically observed epithelialization (using HE and Mason Trichrom staining); (3) Microscopically observed granulation (using HE and Mason Trichrom staining); (4) Microscopically observed collagen deposition (using Mason Trichrom staining); and (5) Microscopically observed capillarization (using CD31 marker: Platelet endothelial cell adhesion molecule (PECAM-1)).

Visual observation indicated absence of any irritation, inflammation, or infection caused by the two sequential applications of the composition; epithelialization occurs faster using the compound compared to control. Higher level of granulation, collagen deposition and capillarization is observed microscopically.

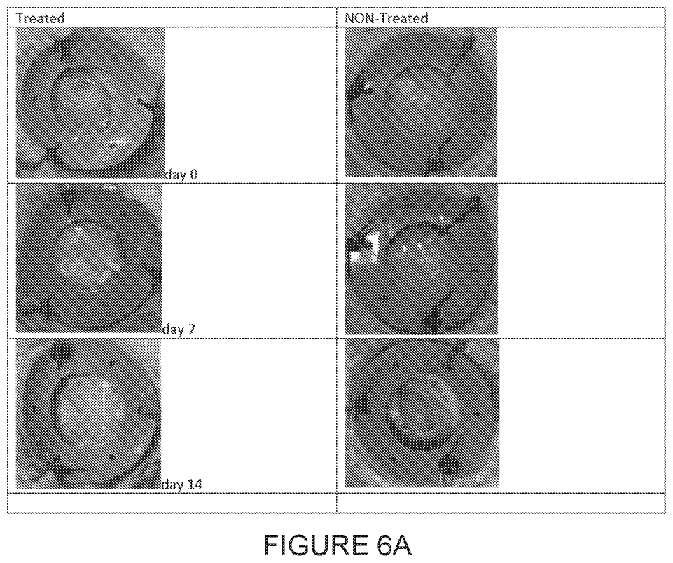

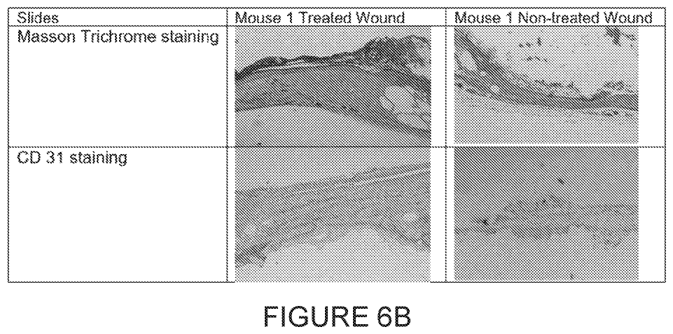

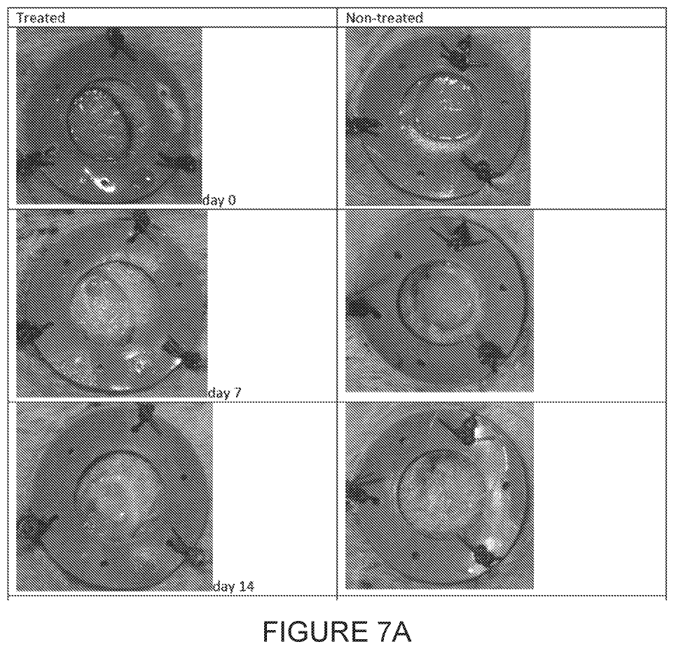

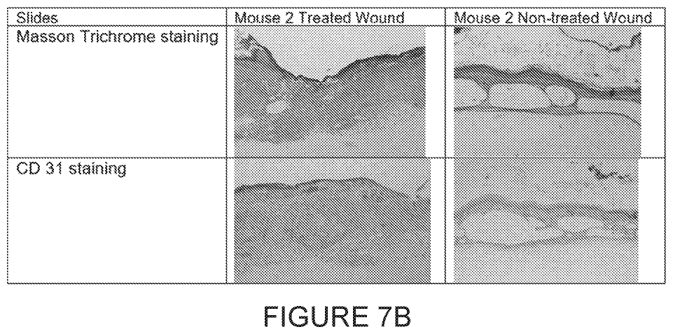

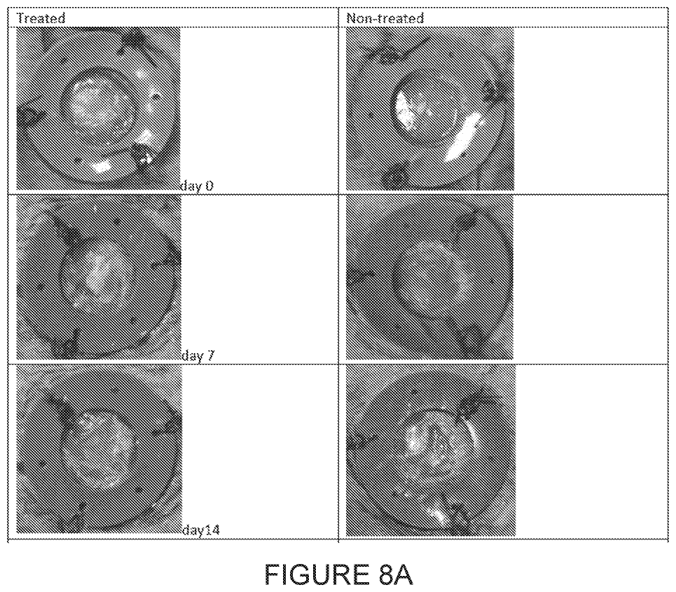

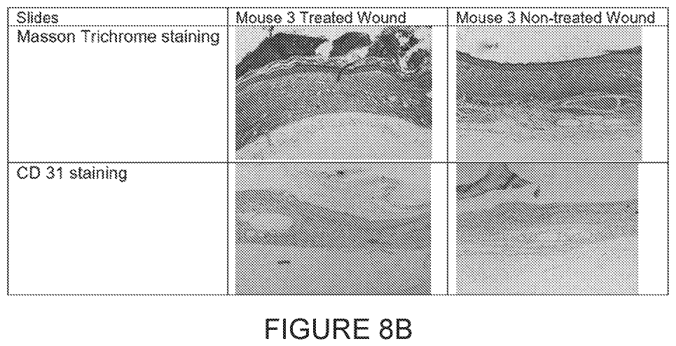

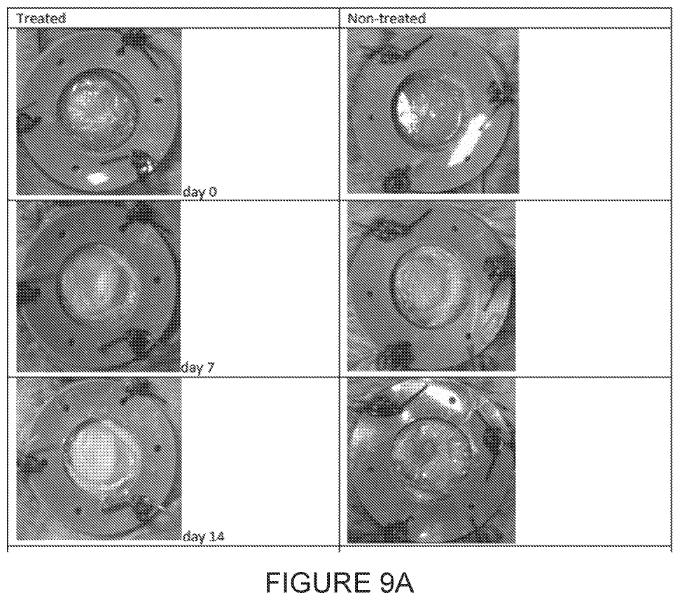

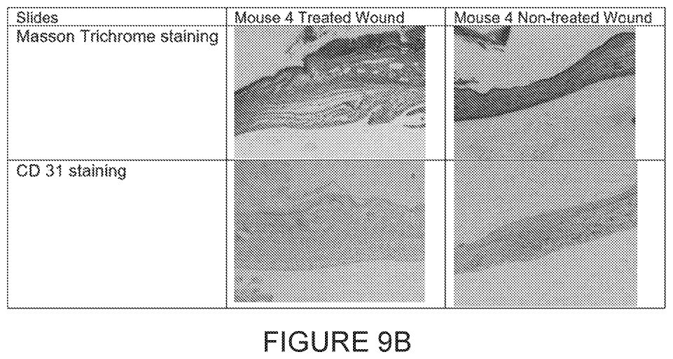

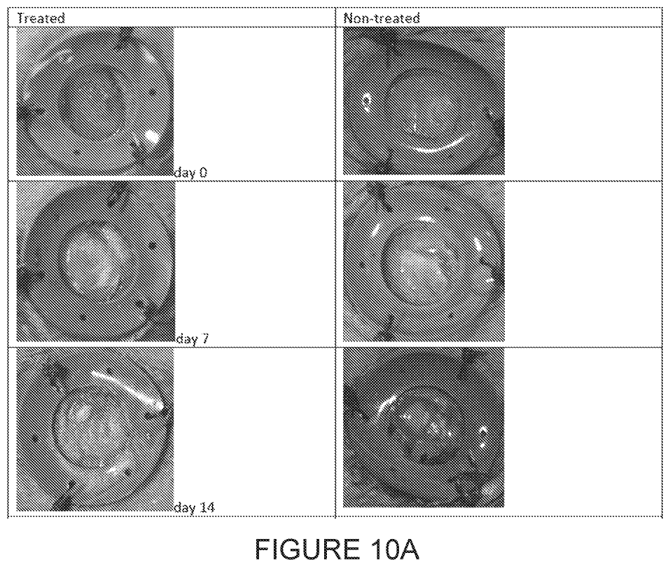

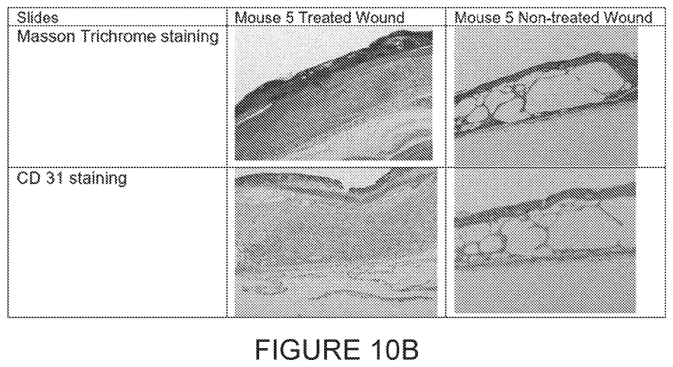

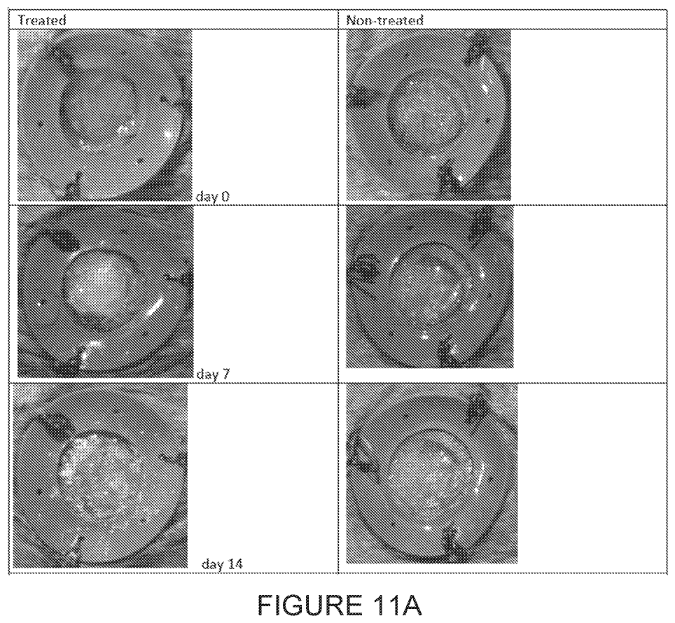

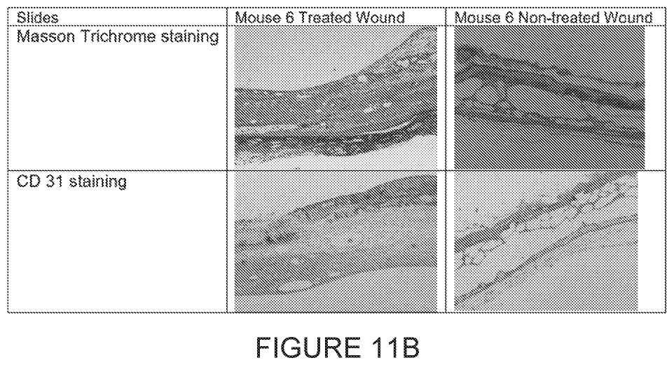

The visual figures of the splinted treated and non-treated wounds indicate visibly an accelerated healing of PEC2 treated wounds compared to non-treated wounds (See FIGS. 6A (mouse 1), 7A (mouse 2), 8A (mouse 3), 9A (mouse 4), 10A (mouse 5), and 11A (mouse 6)). Microscopic staining of treated and non-treated skin tissue sections at wound site demonstrate after 14 days larger thicknesses for epithelial, as well as, the granular layer in PEC2 treated wounds. More collagen deposition is apparent in the treated wounds (see FIGS. 6B (mouse 1), 7B (mouse 2) 8B (mouse 3), 9B (mouse 4) 10B (mouse 5, and 11B (mouse 6)). The CD31 marker indicates further overall enhanced vascularization in the granular tissue layer of PEC2 treated wounds compared to non-treated controls. Table 1 describes the obtained numerical averages for aranular layer thickness and that of the epithelial layer thickness measured in micro meters at the wound site that was treated with the compositions described herein versus the non-treated control in each tested diabetic mouse.

TABLE 1
Granular and Epithelial Layer Thickness

| Mouse 1 | Gender F Age 13 W | Glucose level 299 mg/dl |
|---|---|---|
| Wound | Granular layer thickness | Epithelial layer thickness |
| Non-treated | 29 | 12 |
| Treated | 203 | 47 |
| Mouse 2 | Gender M Age 22 W | Glucose level 313 mg/dl |
| Wound | Granular layer thickness | Epithelial layer thickness |
| Non-treated | 70 | 29 |
| Treated | 233 | 87 |
| Mouse 3 | Gender M Age 22 W | Glucose level 341 mg/dl |
| Wound | Granular layer thickness | Epithelial layer thickness |
| Non-treated | 116 | 23 |
| Treated | 186 | 35 |
| Mouse 4 | Gender F Age 12 W | Glucose level 345 mg/dl |
| Wound | Granular layer thickness | Epithelial layer thickness |
| Non-treated | 64 | 23 |
| Treated | 174 | 23 |
| Mouse 5 | Gender F Age 15 W | Glucose level 323 mg/dl |
| Wound | Granular layer thickness | Epithelial layer thickness |
| Non-treated | 47 | 17 |
| Treated | 232 | 41 |
| Mouse 6 | Gender F Age 15 W | Glucose level 199 mg/dl |
| Wound | Granular layer thickness | Epithelial layer thickness |
| Non-treated | 29 | 17 |
| Treated | 209 | 29 |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations)). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition comprising an extracellular matrix (ECM) or ECM protein isolated from a culture of small mobile stem (SMS) cells, wherein said ECM or ECM protein, preferably comprises at least one induced cross-link, which has been induced by exposure to a cross-linking agent or a cross-linking technique e.g., radiation, chemical, mechanical, or temperature.

2-38. (canceled)
